(19) United States
(12) Patent Application Publication
(10) Pub. No.: US 2012/0277774 A1
(43) Pub. Date: Nov. 1, 2012

(54) Method and Apparatus for Vascular Anastomosis

(75) Inventor: Lifei Guo, Brookline, MA (US)

(73) Assignee: THE BRIGHAM AND WOMEN'S HOSPITAL, INC., Boston, MA (US)

(21) Appl. No.: 13/509,153

(22) PCT Filed: Nov. 12, 2010
(86) PCT No.: PCT/US10/56513

§ 371 Date: Jul. 24, 2012

Related U.S. Application Data
(60) Provisional application No. 61/260,969, filed on Nov. 13, 2009.

(51) Int. Cl. A61B 17/11

(52) U.S. Cl. 606/153

(57) ABSTRACT

Methods and apparatus can be used for anastomosis, and more specifically, for joining two vascular vessels, e.g., arterial or venous vessels or the like, using a stent.

### CROSS REFERENCE TO RELATED APPLICATION

The present application claims priority from U.S. Provisional Patent Application Ser. No. 61/260,969, filed on Nov. 13, 2009, the disclosure of which is incorporated herein by reference in its entirety.

### FIELD OF THE INVENTION

The present disclosure is directed to methods and apparatus for vascular anastomosis, and more particularly for methods and apparatus for joining blood vessels or other tissue structures using a stent-based device.

### BACKGROUND INFORMATION

Microsurgical procedures are often a preferred or necessary surgical modality for reconstructing difficult defects. They often require anastomosis (joining) of small vascular vessels, e.g., veins and arteries, which can be damaged or severed. Such vessels typically have a diameter between about 0.5 and 1.5 mm.

Anastomoses are typically hand-sewn under a microscope using about 8-10 sutures, and the distance between adjacent sutures is typically about 0.3 to 0.4 mm. An exemplary sutured anastomosis with a double opposing vascular clamp stabilizing the two vessel ends is shown in FIG. 1.

Microsurgical procedures tend to be time and resource consuming, technically demanding, and prone to complications. Such procedures often include repair of several damaged blood vessels. For example, there can be a need for about 2-4 anastomoses in a single microsurgical procedure. A set of anastomoses performed by a skilled surgeon can thus take between about 60-120 minutes (e.g., each anastomosis can require between about 20-40 minutes to perform). Technical imperfections arising from the anastomosis can lead to local thrombosis and eventual loss of the transferred tissue. The overall success of surgical procedures can often depend on the quality of the blood vessel anastomosis procedures.

Microsurgery is essential to complex reconstructions, especially when covering large and difficult composite tissue defects. Accordingly, reconstructive cases that require microsurgical approach are generally referred to specialized centers for handling by particularly skilled surgeons, in both civilian and military settings.

Over the years, various advances have been made for simplifying or improving the quality of microvascular anastomosis, including development of various techniques and devices that facilitate hand-suturing of vessels. One device that can be used is the microvascular anastomotic coupler system by Synovis® (Synovis MCA, Birmingham, Ala.), which is described in K. Nakayama et al., “A simple new apparatus for small vessel anastomosisi,” Surgery 52, pp. 918-931 (1962). This coupler system is shown in use in FIG. 2A, and a single coupler device is shown in FIG. 2B. The coupler device shown in FIG. 2B includes a high-density polyethylene ring and six stainless steel pins with corresponding holes between them. Two such devices are used in a typical anastomosis. In use, the end of a vessel to be joined is passed through the center of one ring. The end of the vessel protruding through the ring is everted such that it covers the distal side of the ring. The six pins on the ring impale the vessel walls and hold the end of the vessel in a spread-apart position. A second coupler device is attached to the end of the second vessel to be joined in the same manner. The rings can then be mechanically coupled by inserting the pins of each ring into the corresponding holes of the opposite ring. This coupling also presses the everted ends of the vessels together between the coupled rings to create a secure vascular anastomosis.

Arteries tend to have thicker and stiffer vessel walls than veins, and thus can be much harder to evert, the Synovis® coupler system has been used predominantly with venous anastomoses. Although it has been used occasionally for arterial anastomosis, it is commonly referred to as a venous coupler. In addition, the apparatus used to deploy the coupler rings in the Synovis® coupler system is cumbersome and difficult to operate in a confined space, which is often required in vascular anastomoses. This system also requires an advanced degree of skill to use. Nevertheless, the Synovis® coupler system can shorten the time needed to perform many microvascular anastomoses, and can also decrease the likelihood of forming an undesirable venous thrombosis.

Accordingly, there is a need for an improved method and apparatus for performing rapid venous and arterial anastomoses. Moreover, there is a need for such methods and apparatus that can be performed by a practitioner having a lesser degree of skill than that required for conventional vascular anatomoses.

### SUMMARY OF THE INVENTION

The present invention relates to methods and apparatus for anastomosis, and more specifically, to such methods and apparatus for joining two vascular vessels, e.g., arterial or venous vessels or the like, using a stent.

Embodiments of the present invention include expandable stents configured to be placed within two vessel ends to be joined, such that the stent can be configured to expand after being placed within the vessel ends to facilitate maintaining of the vessel ends in a coapted arrangement. Expanding the stent after placing it within the vessels can facilitate securing or affixing of the stent to the inside walls of the vessels, thereby inhibiting or preventing movement of the stent relative to the vessel walls. The stent can be expanded by using one or more balloons provided within the stent, a tensioned filament, string, or wire, or the like. The stent can also be formed using a shape-memory material, such that the stent in a compacted state can expand when heated up to body temperature after insertion in the vessel ends.

Self-expanding stents can also be used, where such stents can be held in a compacted or compressed state using one or more retainers, and then allowed to expand after being placed in the vessel ends by releasing, cutting, or otherwise deactivating the retainers. The self-expanding stents can include one or more retainers provided at or near each end of the stent, such that each end of the stent can be expanded at different times. In this manner, one end of the stent can be placed in a first vessel end and expanded to anchor it within the first vessel. The unexpanded end can then be placed into an opening of the second vessel end, and expanded after the two vessel ends are coapted to secure the stent within the second vessel and hold the ends of the vessels together through frictional forces and mechanical anchoring of the stent against the inner vessel walls. A tissue adhesive or other substance can optionally be applied over the joined tissue portions to improve the adherence and/or sealing of the vessels.

A sleeve can also be provided over the tissue junction and around the inserted stent, such that the vessel walls are held between the inner expanded stent and the outer sleeve. The sleeve can be substantially cylindrical in shape, and can be provided in a hinged configuration that includes two sleeve portions pivotally attached to one another e.g., pivoting along a line that is substantially parallel to the longitudinal axis of the cylinder. The hinged sleeve can include a securing arrangement configured to hold the two sleeve portions in the shape of a hollow cylinder when the sleeve portions are placed around the vessels in a closed configuration. In certain embodiments, the sleeve can be formed using a deformable material that can be pressed in place around the coapted vessels and supporting stent. The inner diameter of the sleeve can be constant, or it can vary along the longitudinal axis of the sleeve. For example, the end regions of the sleeve can have different internal diameters to facilitate joining of two vessels having different sizes and/or or wall thicknesses. The central portion of the sleeve can also be provided with a larger internal diameter than that of the end portions. Such a larger central diameter can better accommodate the coapted ends of the vessels when the sleeve is placed around the joined vessels.

In further embodiments, a kit includes a plurality of stents, which can have different lengths and/or expanded diameters, to facilitate joining of a plurality of vessels in a microsurgical procedure. A plurality of sleeves having different lengths and/or diameters can also be provided that can be selected to fit over a particular stent and around vessels having a particular wall thickness.

Further embodiments of the invention can facilitate formation of side-to-end grafts, e.g., for affixing the end of a vessel to an opening in a wall of a larger vessel or a biological organ. For example, a stent can be provided that includes a proximal end provided in a form of a conventional expandable stent and configured to be inserted into and affixed to an end of a first vessel. A distal end of the stent can be provided with a plurality of extensions configured to extend outward from the longitudinal axis of the stent in the expanded state. The extensions can be held in a compressed or compacted state before the distal end is inserted through a wall of a larger vessel or organ, using one or more retainers. The retainers can be cut, released, or otherwise deactivated after the distal end of the stent is inserted through the wall of the larger vessel or organ, allowing the extensions to expand and press against the inner side of the wall, thereby securing the first vessel to the wall. The extensions can have different lengths, e.g., to allow the expanded extensions to better conform to the shape of the inner wall of the larger vessel or organ.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### BRIEF DESCRIPTION OF THE DRAWINGS

Further objects, features and advantages of the present invention will become apparent from the following detailed description taken in conjunction with the accompanying figures showing illustrative embodiments.

FIG. 1 is an image of a conventional hand-sewn anastomosis and a clamp holding the vessel ends in place.

FIGS. 2A and 2B are images of a prior art microvascular coupler system in use, and a single coupler.

FIGS. 3A-3E are schematic illustrations of a balloon-expandable stent being used to form an anastomosis in accordance with an embodiment of the present invention.

FIGS. 4A-4D are schematic illustrations of a stent expandable using two independent balloons being used to form an anastomosis in accordance with another embodiment of the present invention.

FIGS. 5A and 5B are schematic illustrations of a tension-expandable stent being used to form an anastomosis in accordance with a further embodiment of the present invention.

FIGS. 6A and 6B are schematic illustrations of a self-expanding stent in an expanded and a compacted state, respectively.

FIGS. 7A-7C are schematic illustrations of the self-expanding stent shown in FIGS. 6A and 6B being used to form an anastomosis in accordance with yet another embodiment of the present invention.

FIGS. 8A and 8B are schematic illustrations of a sleeve that can be placed around the anastomosis in accordance with embodiments of the present invention.

FIGS. 9A and 9B are schematic illustrations of an exemplary hinged sleeve that can be used with embodiments of the present invention.

FIG. 10 is an exemplary illustration of a bypass graft that can be joined using embodiments of the present invention.

FIGS. 11A and 11B are schematic illustrations of an exemplary self-expanding stent that can be used to form a side-to-end graft in accordance with another embodiment of the present invention.

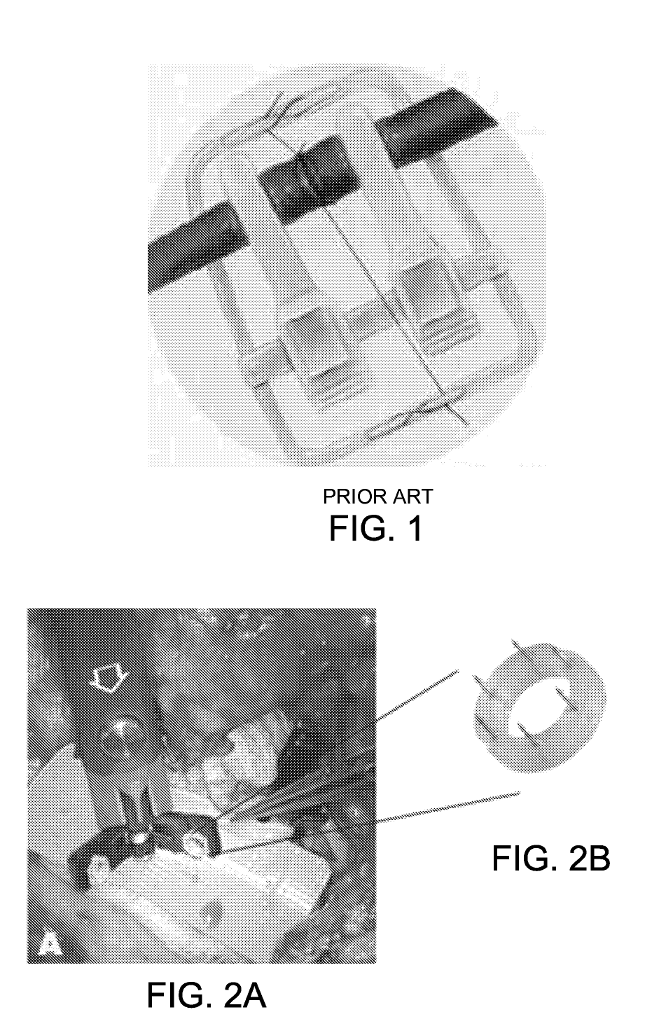

While the present invention will now be described in detail with reference to the figures, it is done so in connection with the illustrative embodiments and is not limited by the particular embodiments illustrated in the figures. It is intended that changes and modifications can be made to the described embodiments without departing from the true scope and spirit of the present invention as described herein.

### DETAILED DESCRIPTION

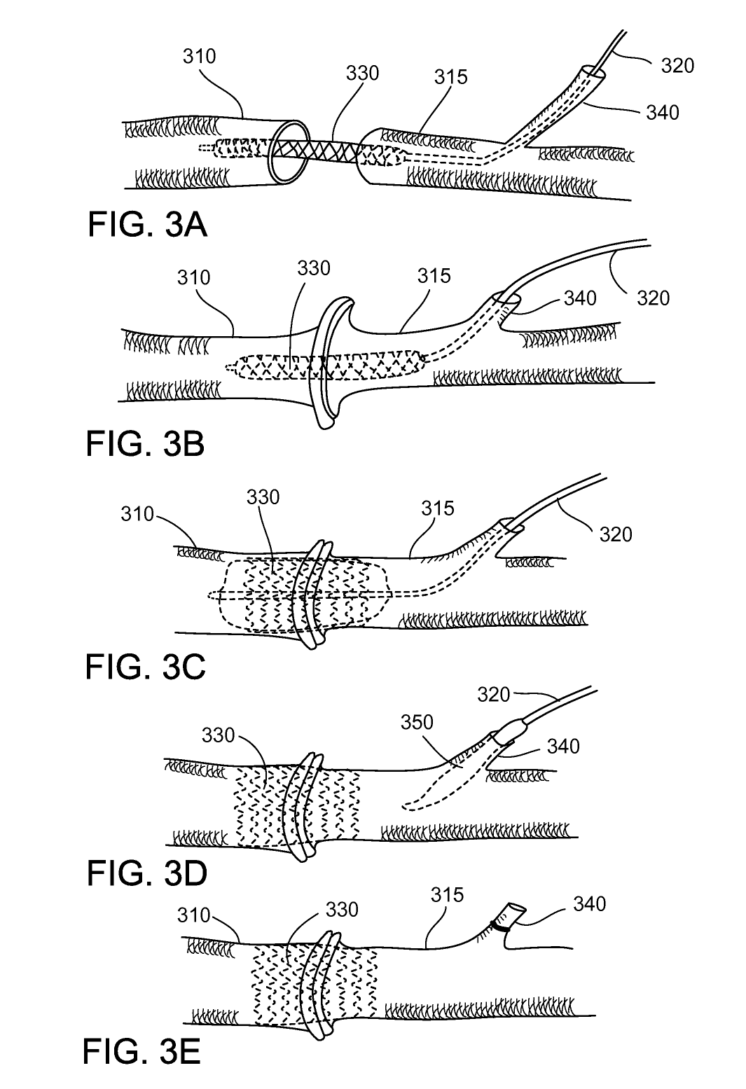

An exemplary method and apparatus for performing an anastomosis are shown in FIGS. 3A-3E. In FIG. 3A, the ends of two vessels 310, 315 to be joined are shown. A catheter 320 or other device that includes a balloon-deployable stent 330 is introduced through a side branch 340, such that the stent 330 passes through both vessel ends. The ends of the vessels 310, 315 are then coapted against each other over the unexpanded stent 330, as shown in FIG. 3B. The stent 330 is then expanded, as shown in FIG. 3C, and the catheter 320 with the deflated balloon apparatus 350 can then be withdrawn through the sidebranch 340, as shown in FIG. 3D. FIG. 3E shows the side branch 340 being sealed off after the catheter 320 and the balloon 350 are withdrawn.

A typical microvascular vessel 310, 315 that can require anastomosis during microsurgical procedures can have an outer diameter between about 1 mm and 4 mm. The expanded diameter or size of the stent 330 can be slightly larger than the diameter or size of the central lumen of the un-distended vessels 310, 315 being joined. This size difference can provide several advantages. For example, this expanded size can prevent the coapted vessel ends from being separated by providing frictional resistance to motion between the stent 330 and the interior surfaces of the vessels 310, 315. The larger stent size can also create an anastomosis that is larger than the original vessel 310, 315, thereby providing a larger lumen where the vessel ends are joined and reducing a likelihood of anastomotic failure by thrombosis.

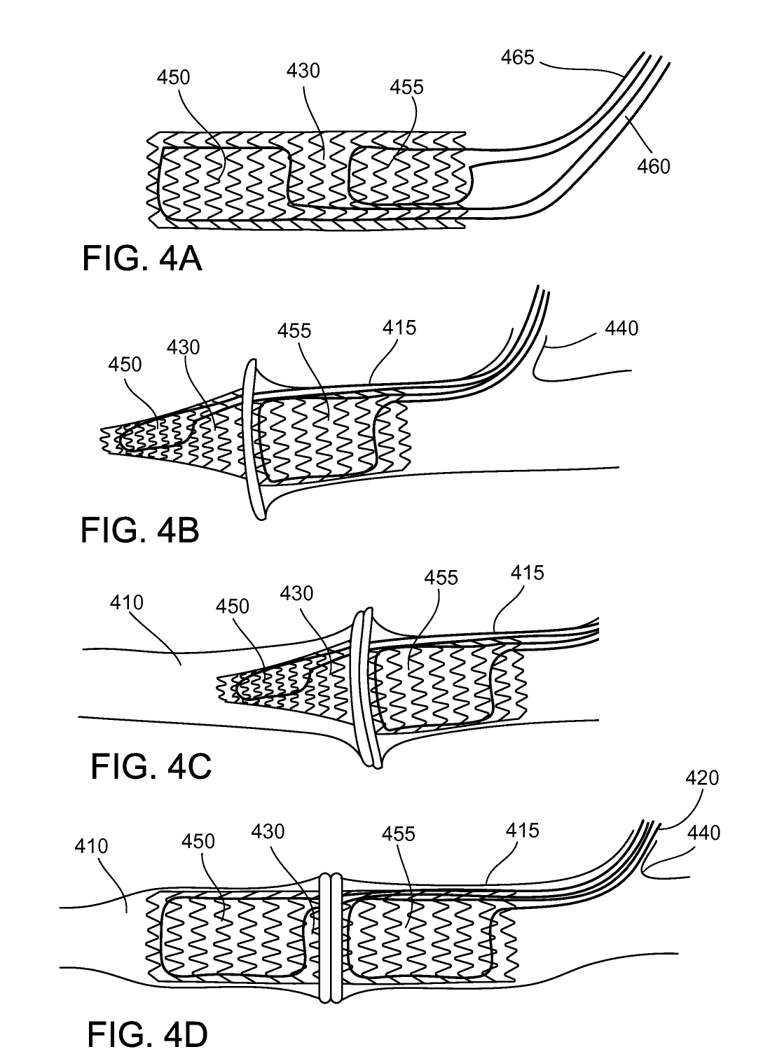

In a further embodiment, an expandable stent 430 can be provided that includes two balloons 450, 455, with one balloon 455 located within a proximal end of the stent 430, and a second balloon 450 located within a distal end of the stent 430 as shown in FIG. 4A. Each balloon 450, 455 can be in communication with a separate lumen 460, 465 that can be provided within a catheter, such that they can be independently inflated and/or deflated. The exemplary two-balloon stent 430 shown in FIG. 4A can be introduced between the ends of two vessels to be joined, similar to the configuration shown in FIG. 3A. The proximal balloon 455 can then be inflated to expand the proximal end of the stent 430 within the end of a first vessel 415, as shown in FIG. 4B. This expansion can facilitate anchoring of the stent 430 within the first vessel end. The end of a second vessel 410 to be joined can then be placed over the unexpanded distal end of the stent 430 and coapted against the end of the first vessel 415, as shown in FIG. 4C. Providing an unexpanded end of the stent 430 that protrudes from and is anchored to the end of the first vessel 415 can facilitate alignment and placement of the end of the second vessel 410. The distal balloon 450 can then be inflated to expand the distal end of the stent 430 within the second vessel 410 while the vessel ends are coapted, as shown in FIG. 4D. The balloons 450, 455 can then be deflated, and the catheter 420 used to deliver the balloons 450, 455 and stent 430 can then be withdrawn and the sidebranch 440 closed off, e.g., in a procedure similar to that shown in FIGS. 3D and 3E.

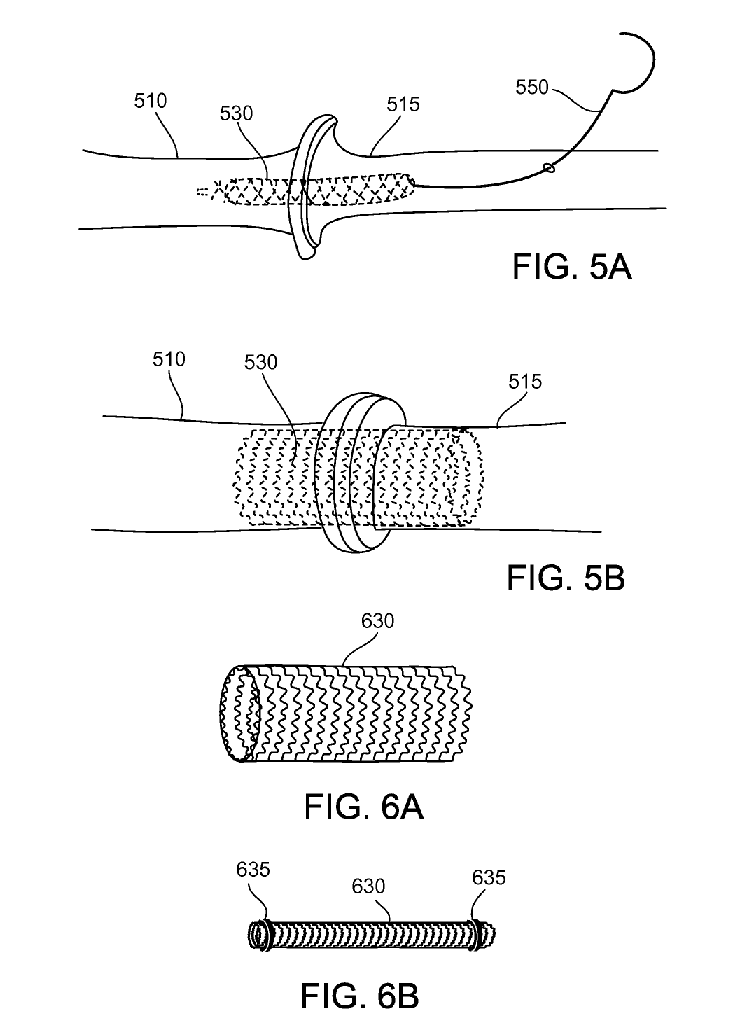

Balloon deployment systems for expanding stents can be too large for anastomosis of certain smaller vessels. Accordingly, in a further embodiment of the present invention shown in FIG. 5A, a stent 530 having a retraction-based deployment arrangement 550 can be provided through the ends of two coapted vessels 510, 515 to be joined. The deployment arrangement 550 can include, e.g., a string, wire, filament, or the like, where the stent 530 can be configured to expand when a tension is applied or increased on the filament, string, or wire, e.g., by pulling on the filament, string, or wire. The stent 530 can be placed within the ends of the vessels 510, 515 using any conventional procedure. This stent 530 can then be expanded by activating the deployment arrangement 550, e.g., by pulling the filament, string, or wire, as shown in FIG. 5B. The deployment arrangement 550, e.g., a filament, string or wire, which can be attached to a fine microsurgical needle, can pass through a side branch or a wall of the vessel 515 further back from the end thereof. This filament, string, or wire can then be removed, e.g., cut off, leaving the expanded stent 530 in place against the coapted vessel ends to form the anastomosis.

In a further embodiment, a self-expanding stent arrangement can be used to form a vascular anastomosis. For example, a stent 630 can be provided having an expanded form such as that shown in FIG. 6A. The stent 630 can be configured to be elastically compressed into a collapsed shape, such as the narrower cylindrical shape illustrated in FIG. 6B. The stent 630 can be constrained in the collapsed shape using, e.g., one or more retainers 635. The retainer 635 can be a stitch, which can circumscribe the collapsed stent 630, a small hook, a small sleeve, or the like. The retainers 635 can be configured to be easily releasable, e.g., by cutting or removing a stitch, or unhooking a hook. Upon releasing the retainer 635, the stent 630 can be configured to relax to the expanded shape shown in FIG. 6A. The material and structure of the stent 630 can be selected such that the compression of the stent 630 produces a substantially elastic deformation, facilitating a return to the pre-compressed shape upon release of the retainers 635. For example, the stent 630 can be formed using a metal or metal alloy used to form conventional stents, including conventional self-expanding stents, where heat treatment and/or structural parameters of the stent 630 can be selected to provide the elastic deformation.

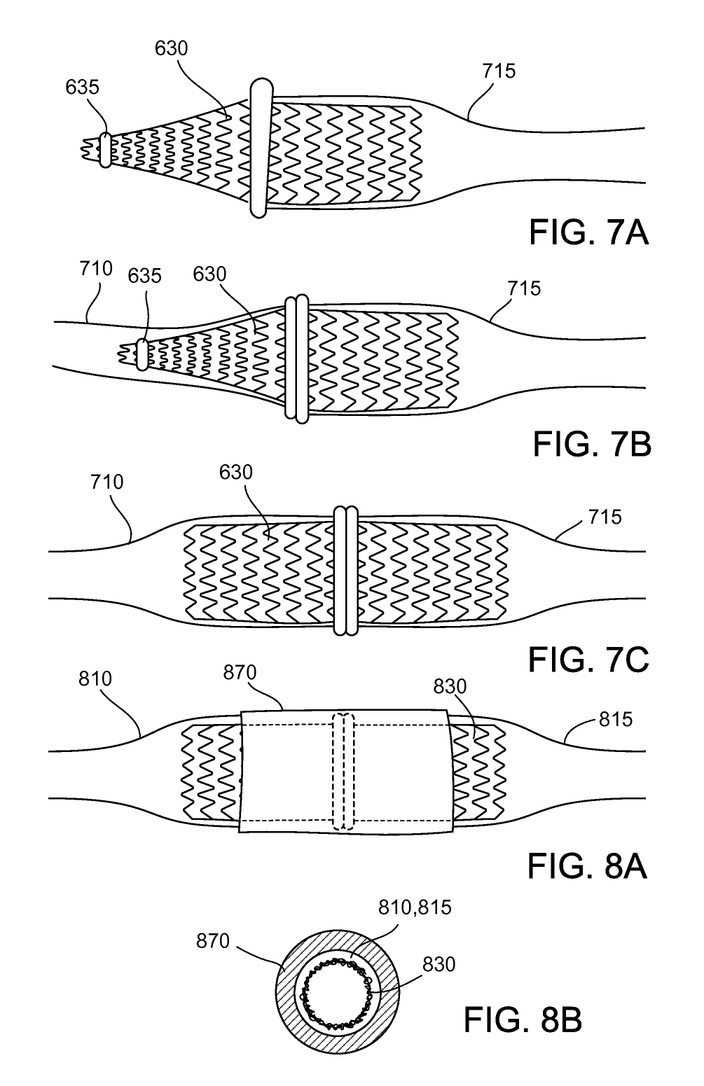

In one embodiment, a self-expanding stent 630 is provided, such as that shown in FIGS. 6A and 6B, that includes a retainer 635 provided proximal to each end of the stent 630. A proximal end of the stent 630 can be inserted into the lumen of a first vessel 715 to be joined such that the distal end of the stent 630 protrudes from the end of the first vessel 715. The retainer 635 at the inserted (proximal) end of the stent 630 can then be released to expand the proximal end of the stent 630 within the end of the first vessel 715, as shown in FIG. 7A. The expansion of the proximal end of the self-expanding stent 630 can secure the stent 630 within the first vessel 715 by pressing outwardly against the inner surface of the vessel 715 when in the expanded state. The retainer 635 can be released from the proximal end of the stent 630 using, e.g., a needle, hook, or the like, which can be inserted with the stent 630 from the open vessel end and brought out through a wall or a sidebranch of the first vessel 715 near the proximal end of the stent 630 and subsequently withdrawn.

The unexpanded distal end of the stent 630, protruding from the end of the first vessel 715, can then be inserted into an open end of a second vessel 710 to be joined, as shown in FIG. 7B. The two vessel ends can then be coapted by pressing the ends of the two vessels 710, 715 towards each other over the stent 630. The retainer 635 at the distal end of the stent 630 can then be released to provide an anastomosis, shown in FIG. 7C, that includes an expanded stent 630 holding the vessel ends together. Such a procedure can provide a local lumen size where the vessel ends are joined that is slightly larger than the lumen size of the relaxed vessels 710, 715 away from the anastomosis.

The size of the expanded stent 630 can be selected to be slightly greater than the local inner diameter of the vessels 710, 715 being joined. As described above, such a larger stent size can help to anchor the stent 630 against the inner walls of the vessels 710, 715, and improve the quality of the anastomosis by increasing the lumen diameter where the vessels 710, 715 are joined, thus reducing the likelihood that some blockage or thrombosis can occur at the anastomosis site. The size of the stent 630 can be selected based on both the size of the vessels 710, 715 being joined and their type.

For example, arterial vessels tend to have thicker and less elastic walls than comparably-sized venous vessels. A stent for an anastomosis procedure as described herein can be used that has a diameter, e.g., about 10-30% larger than the inside diameter of a relaxed arterial vessel to be joined. This larger stent size can be large enough to provide sufficient force against the inner wall of the vessels to expand them slightly and position the stent firmly with respect to the vessel ends. The stent should not be so large as to tear or otherwise damage the vessel wall when the stent is inserted and expanded.

Venous walls tend to be thinner and more elastic. Accordingly, the size of stents used for anastomosis of venous vessels can be between, e.g., about 20-100% larger than the inner diameter of the relaxed venous vessels to be joined, for the reasons described above with respect to arterial vessels. These relatively larger stent sizes can provide sufficient force against the more elastic venous vessel walls to inhibit movement of the stent relative to the vessels when it is expanded.

A length of the stent can be selected based on the size of the vascular vessels to be joined. For example, the length of a stent can be between about 3× and 8-10× as long as its expanded diameter. Shorter stents can be difficult to place accurately across the ends of the vessels being joined, and also can not provide sufficient frictional support to maintain the anastomosis in an expanded size. A longer stent can form a corresponding longer length of rigidly supported, less flexible vessel that can interfere with adjacent tissues or otherwise be undesirable.

In one embodiment, a stent can be used as described herein that is formed using a biodegradable or absorbable material. Such material can preferably have a functional lifespan, e.g., be capable of mechanically expanding the adjacent vessel walls, that is greater than about 3 weeks when placed in an environment such as a vascular vessel. For example, in certain microsurgical procedures (e.g., reconstructive microsurgery) collateral blood supplies can develop that can accommodate the function of the anastomosized vessel within a few weeks, such that the longevity of the stent and anastomosis beyond a few weeks can not be affect the success of the microsurgical procedure. In certain procedures, such as cardiac, vascular, or transplant surgery, the long term patency of the anastomosis can be important. Accordingly, a biodegradable stent can be preferable for such procedures because the removal of any foreign materials intra-vascularly would facilitate maintenance of the anastomotic patency. Such degradable material can preferably maintain mechanical and structural properties for, e.g., about 3-6 weeks or more for the anastomosed vessel ends to completely heal.

In another embodiment, the stent can be formed using a shape memory material, e.g., a Nitinol alloy or the like. For example, a shape memory alloy can be selected to form the stent such that the stent expands from a compacted state to an expanded state when it is heated from a temperature at or below room temperature to body temperature. Such a stent can be placed within the vessels to be joined as described herein, and it can then expand spontaneously as it heats up within the vessel ends to a predetermined final size.

In further embodiments, a tissue glue, cement, or other adhesive can be used to provide a stronger connection and/or better seal between the vessel ends after the stent has been positioned and expanded as described herein. Such adhesive can be applied over the coapted ends of the vessels, and optionally over a portion of the proximal outer surfaces of the vessels. Application of the adhesive can improve the seal between coapted ends of the joined vessels, and can further add mechanical strength to the junction.

Other substances can be applied to the coapted vessel ends in addition to or instead of an adhesive, such as a growth stimulator, a solution or gel containing stem cells, etc. Such materials can facilitate joining of the vessel ends after they are coapted and held in place by the stent.

In a further embodiment, a sleeve 870 having a substantially cylindrical hollow shape can be provided around the ends of coapted vessels 810, 815 after a stent 830 is placed within them and expanded, e.g., in accordance with any of the various embodiments described herein. Such a sleeve is shown in FIG. 8A. The sleeve 870 can be shorter in length than the stent 830 provided within the joined vessel ends, as illustrated in FIG. 8A, to ensure that the entire length of the sleeve 870 is supported by the internal stent 830. For example, the length of the sleeve 870 can be about half the length of the stent 830, or it can be about 1-3 mm shorter than the stent 830 at each end. Other sleeve lengths can also be selected for particular procedures as appropriate. In general, it can be preferable to provide a sleeve 870 that is shorter than the corresponding stent, such that the entire length of the exterior sleeve 870 is supported by the stent 830 placed inside the vessels being joined.

FIG. 8B is a cross section of the sleeve 870 in FIG. 8A. The wall of the vessel 810, 815 is located between the inside of this sleeve 870 and the outside of the stent 830. The diameter of the sleeve 870 can be selected such that it is slightly smaller than the outside diameter of the joined vessels 810, 815 having an expanded stent 830 within them. For example, the inner radius of the sleeve 870 can be slightly smaller than the sum of the stent radius (in the expanded state) and the thickness of the vessel wall. This exemplary sizing can slightly compress the vessel walls between the inner surface of the sleeve 870 and the outer surface of the stent 830. Such a configuration can provide more support for the vessels 810, 815, facilitate formation of a better seal between the coapted vessel ends, etc. The sleeve diameter is preferably large enough to avoid damaging the vessel walls when placed over the anastomosis, but sufficiently small to press against the outer surface of the vessels 810, 815 to prevent a shift or movement of the sleeve 870 along the vessel axis.

In certain embodiments, the inner diameter of the hollow cylindrical sleeve 870 can vary along the length of the sleeve 870. For example, the inner diameter of the sleeve 870 can be slightly larger in the central region of the sleeve to better accommodate the coapted ends of the vessels 810, 815, which can have a larger diameter than the vessels 810, 815 away from their coapted ends. In another exemplary embodiment, the internal diameter of the central portion of the hollow cylindrical sleeve 870 can be slightly smaller than that of the distal portions of the sleeve 870. Such a narrower diameter can provide further compression of the coapted ends of the vessels 810, 815 to improve the seal between them. In still further embodiments, the inner diameter of the hollow cylindrical sleeve 870 can continuously vary along the axis of the sleeve 870, or one side of the hollow cylindrical sleeve 870 can have an internal diameter that is different than the internal diameter of the other side of the sleeve 870. Such configurations can provide a more uniform fit of the sleeve 870 around the vessels 810, 815 when the vessels 810, 815 have different sizes and/or wall thicknesses.

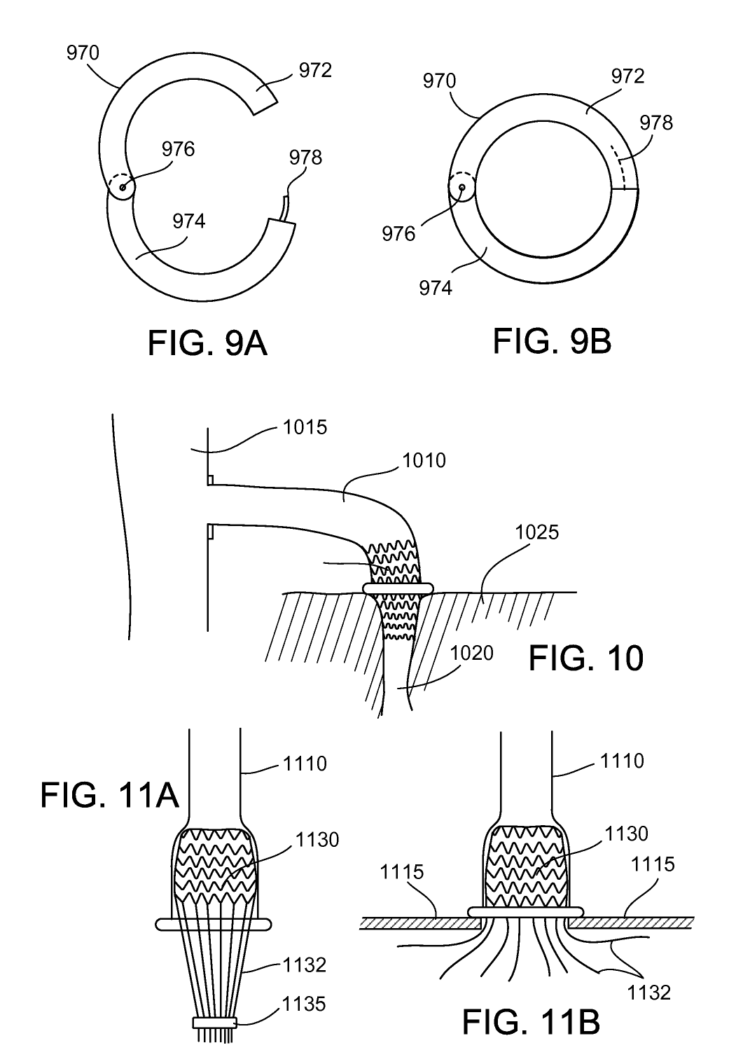

In one embodiment, such a sleeve 970 can be formed in two half-cylinder pieces 972, 974 that can be hinged together. A cross-section of such a hinged sleeve 970 is shown in FIG. 9A. The sleeve hinge 976 shown in FIG. 9 can include, e.g., one or more pivot pins that extend parallel to the axis of the sleeve 970 and allows the two halves 972, 974 of the sleeve 970 to rotate relative to one another. Alternatively, a hinge 976 can be provided by forming a groove or indentation (e.g., a wedge-shaped groove) in the outer surface of a solid sleeve 970 that extends parallel to the axis of the sleeve 970, and providing a single slit through the sleeve thickness opposite this groove. The thinner material at the groove can be flexible, allowing the two sleeve sections 972, 974 to pivot where they are attached at the groove. A hinged sleeve 970 can also be provided that is formed from two half-cylinders 972, 974 that are joined along one edge by a flexible material such as an adhesive tape or the like.

The opening formed by the split side of the sleeve 970 can be placed over the joined vessels, and the sleeve 970 can then be pressed or squeezed closed so that the open edges meet to form a closed cylinder, as shown in cross-section in FIG. 9B. A plurality of pins 978 can be provided along an open edge of a first half 974 of the hinged sleeve 970 such that they penetrate into the edge of the other half 972 of the sleeve 970 or, alternatively, fit into corresponding holes provided in the open edge of the other half 972 of the sleeve 970 when the sleeve 970 is pressed into a closed shape. Insertion of a pin 978 from the lower half 974 of the sleeve 970 into the upper half 972 of the sleeve 970 is shown in FIG. 9B. The pins 978 are preferably sufficiently long to provide a reliable connection between the sleeve edges, but not so long as to interfere with placement of the split sleeve 970 over the anastomosis prior to closing it.

Other arrangements can be used to secure such a sleeve around the anastomosis site. For example, one or more small clips or the like can be provided along the split edges of the sleeve, such that they interlock and hold the sleeve closed when the edges are pressed together. Alternatively, the sleeve can be formed of a deformable material, such that it can be clamped around the anastomosis site and retain a closed shape after placement. For example, a deformable sleeve can be configured to be crimped onto the anastomosis site using a force that is sufficient to deform the sleeve but not so great as to distort the expanded stent within the vessel.

Embodiments of the present invention can be used for various sizes of vessels, including larger vessels that can be joined in vascular or transplant surgery. Such stents, and corresponding sleeves, if used, can be sized according to the exemplary criteria and functions described herein.

Embodiments of the present invention can be used to join vessels to other tissues, e.g., for a coronary bypass graft. An example of a bypass graft is shown in FIG. 10. A section of vein graft 1010 connected to the aorta 1015 can be attached to an opening 1020 in the heart muscle 1025 using an expandable stent 1030. The stent 1030 is shown in an expanded state in FIG. 10. The end of the stent 1030 within the vein graft 1010 can be expanded first and anchored to the end of the graft vessel 1010, as shown in FIG. 7A. The unexpanded distal end of the stent 1030 can then be inserted into the opening 1020 in the heart muscle 1025 and expanded, e.g., by releasing a retainer provided at the distal end as described herein. The stent 1030 can be shaped to provide sufficient anchoring and conformance to the tissue structures being joined. For example, the distal end of the stent 1030 shown in FIG. 10 can be tapered and/or narrower than the proximal end, to provide a good fit within the opening 1020 in the heart muscle 1025, which can be less elastic or stretchable than the venous graft tissue. An adhesive or other substance can be applied at the junction of the venous graft 1010 and the heart muscle 1025 to improve the mechanical stability and/or provide a better seal of the coapted tissues.

In another embodiment, embodiments of the present invention can be used to form a side-to-end graft. For example, an expandable stent 1130 such as that shown in FIG. 11A can be anchored within a first vessel 1110 to be joined to a wall 1115 of a larger vessel, an organ, or the like, by inserting and expanding the proximal portion of the stent 1130 within the vessel end, e.g., using one of the exemplary techniques and arrangements described herein. The distal portion of the stent 1130 can include a plurality of wires 1132 or other extensions that can be held in a compressed form using one or more retainers 1135, as shown in FIG. 11A. The extensions 1132 can be configured to expand or splay out away from the longitudinal axis of the stent 1130 when they are unconstrained, e.g., when the retainer 1135 is removed, cut, or otherwise released. The extensions 1132 can be configured to expand into the larger vessel, and they can be shorter in length than the larger vessel diameter, e.g., to avoid possible interference between the extensions 1132 and the larger vessel walls 1115 when expanding this end of the stent 1130. A pull string or wire (not shown) can also be provided, e.g., using a surgical needle or the like, and can be utilized to tent or expand the opposing vessel wall while being retrieved to allow the splaying extensions 1132 to turn and be oriented in a preferred direction with respect to the axis of larger vessel. For example, the extensions 1132 can be provided with non-uniform lengths, such that when expanded within the larger vessel, longer ones of the extensions 1132 extend along the larger vessel wall 1115 in a direction of the larger vessel's longitudinal axis, and shorter ones of the extensions 1132 extend perpendicular or at other angles with respect to the larger vessel's longitudinal axis. Such a configuration facilitates better shape conformity between the splayed extensions 1132 and the wall 1115 of the larger vessel.

The distal end of the stent 1130 can be inserted through a hole provided in a wall 1115 of a second vessel or other tissue layer to which the first vessel 1110 is to be joined, and the end of the first vessel 1110 can be coapted against the wall 1115 of the second (larger) vessel or tissue surface. The retainer 1135 at the distal end of the stent 1130 can be released using any appropriate mechanism, allowing the extensions 1132 at the distal end of the stent 1130 to expand up against the inner surface of the second vessel or tissue wall 1115. For example, the extensions 1132 at the distal end of the stent 1130 can curve slightly back towards the proximal end when allowed to expand, e.g., as shown in FIG. 11B, which can facilitate a joining and better seal between the tissue surfaces being joined. An adhesive and/or other substance can also be applied to the portions of the joined tissue to improve the seal and/or attachment between them.

The exemplary methods and devices described herein can facilitate improved anastomosis and anastomosis of smaller vessels, including both arterial and venous vessels. Embodiments of the present invention can facilitate faster and/or more reliable anastomosis of vascular vessels and other similar tissue structures. Such anastomosis can also require less skill than hand suturing, and thereby can be performed by a larger number of surgeons in a greater number of facilities.

Embodiments of the present invention can provide a mechanical support in the area of the joined vessels using a stent, and can also dilate the lumen in the joined area to reduce the likelihood of collapse, blockage, or thrombosis of the anastomosis.

In further embodiments, a kit is provided that includes a plurality of stents having different lengths and/or expanded diameters. The size increments can be provided at suitable intervals to enable anastomosis of a variety of vessel sizes. The kit can further include a tissue adhesive or the like. A plurality of sleeves as described herein can also be provided in the kit. The sleeves can also be provided in different lengths and diameters so that they fit over the coapted vessels having various wall thicknesses that have been expanded by stents having a range of expanded diameters, as described herein.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. An apparatus for anchoring a vascular vessel to an anatomical structure, comprising:
an expandable stent configured to be inserted at least partially within an end portion of the vascular vessel; and
a first anchoring arrangement configured to affix a proximal portion of the stent to the vascular vessel,
wherein a distal portion of the stent is configured to be affixed to the anatomical structure.

2. The apparatus of claim 1, wherein the anatomical structure comprises a further vascular vessel, and wherein the first anchoring arrangement is further configured to affix the distal portion of the stent to the second anatomical structure.

3. The apparatus of claim 2, wherein the first anchoring arrangement comprises a balloon arrangement provided at least partially within the stent.

4. The apparatus of claim 2, wherein the first anchoring arrangement comprises at least one of a filament, a string, or a wire that is provided in communication with the stent.

5. The apparatus of claim 1, further comprising a second anchoring arrangement, wherein:
the second anatomical structure comprises a further vascular vessel;
the first anchoring arrangement comprises a first balloon arrangement provided at least partially within the proximal portion of the stent; and
the second anchoring arrangement comprises a second balloon arrangement provided at least partially within the distal portion of the stent.

6. The apparatus of claim 1, wherein the stent is a self-expanding stent, and the first anchoring arrangement comprises a first retainer arrangement configured to maintain at least a portion of the stent in a contracted configuration before the stent is inserted at least partially into the end portion of the vascular vessel.

7. The apparatus of claim 6, further comprising a second retainer arrangement, wherein the first retainer arrangement is provided at a proximal portion of the stent and the second retainer arrangement is provided at a distal portion of the stent, and wherein the second retainer arrangement is configured to maintain the distal portion of the stent in a contracted configuration before the distal portion of the stent is inserted at least partially into the anatomical structure.

8. The apparatus of claim 2, further comprising a sleeve arrangement configured to be placed around at least a portion of the stent and around the end portion of the vascular vessel.

9. The apparatus of claim 8, wherein the sleeve arrangement comprises a first sleeve portion pivotally connected to a second sleeve portion, wherein the first and second sleeve portions are configured to form a hollow cylindrical structure when they are arranged in a closed position.

10. The apparatus of claim 9, further comprising a securing arrangement configured to maintain the sleeve arrangement in a hollow cylindrical configuration when the first and second sleeve portions are arranged in the closed position.

11. The apparatus of claim 8, wherein a length of the sleeve arrangement is smaller than a length of the stent.

12. The apparatus of claim 1, wherein the distal portion of the stent comprises a plurality of extensions configured to expand away from a longitudinal axis of the stent and affix the distal portion of the stent to the anatomical structure.

13. The apparatus of claim 12, further comprising a second retainer arrangement, wherein the second retainer arrangement is configured to maintain the distal portion of the stent in a contracted configuration before the distal portion of the stent is inserted at least partially into the anatomical structure.

14. The apparatus of claim 12, wherein at least two of the extensions have different lengths.

15. A method of anchoring a vascular vessel to an anatomical structure, the method comprising:
inserting an expandable stent configured at least partially within an end portion of the vascular vessel;
affixing a proximal portion of the stent to the vascular vessel; and
affixing a distal portion of the stent to the anatomical structure.
